(19) United States
(12) Patent Application Publication
(10) Pub. No.: US 2010/0233194 A1
(43) Pub. Date: Sep. 16, 2010

(54) TREATMENT OF NEOVASCULAR OCULAR DISEASE STATES

(76) Inventors: Jean-Philippe Combal, Dijon (FR); Elisabeth Latour, Paris (FR); Pierre Belichard, Paris (FR)

Correspondence Address: BUCHANAN, INGERSOLL & ROONEY PC, POST OFFICE BOX 1404, ALEXANDRIA, VA, 22313-1404, US

(21) Appl. No.: 12/593,758

(22) PCT Filed: Mar. 27, 2008
(86) PCT No.: PCT/EP08/02426

§ 371 Date: Mar. 31, 2010

(30) Foreign Application Priority Data
Mar. 30, 2007 (EP) 07 36 0012.4

(51) Int. Cl. A61K 39/00; A61K 38/13; A61K 31/56; A61P 27/02

(52) U.S. Cl. 424/184.1; 514/11; 514/171

(57) ABSTRACT

Compositions and regime or regimen for inhibiting unwanted ocular angiogenesis include the treatment of ocular neovascularization by administration of anti-angiogenesis agents, e.g., agents that inhibit VEGF, in combination with a second ocular therapy.

The present Invention relates to compositions and methods for inhibiting unwanted angiogenesis of ocular tissues, and therefore for preventing and/or treating ocular diseases involving angiogenesis process. More specifically, it relates to compositions and methods for preventing and/or treating neovascularization of ocular tissues, using agents that inhibit VEGF in combination with a second therapy.

Angiogenesis, also called neovascularization, is a fundamental process whereby new blood vessels are formed. Under normal physiological conditions angiogenesis is highly regulated and essential for reproduction, embryonic development, tissue repair and wound healing (for a review see Carmeliet, 2005, Nature, 438, 932-936). However angiogenesis also occurs under various pathological conditions, including tumor growth and metastasis, inflammatory disorders such as rheumatoid arthritis, psoriasis, osteoarthritis, inflammatory bowel disease, Crohn's disease, ulcerative colitis and others, and ocular neovascularization such as in diabetic retinopathy, age related macular degeneration (AMU) and various other eye diseases (see for example Folkman, 1995, Nat. Med., 1, 27-31). Actually, angiogenesis occurs in response to various proangiogenic stimuli like growth factors, cytokines and other physiological molecules as well as other factors like hypoxia and low pH (Folkman and Shing, 1992, JBC, 267, 10931). The angiogenic cascade for development of new blood vessels requires the cooperation of a variety of molecules that regulate necessary cellular processes such as extracellular matrix (ECM) remodelling, invasion, migration, proliferation, differentiation and tube formation (Brooks, 1996, Eur. J. Cancer, 32A, 2423). After an initiation phase proangiogenic molecules like VEGF, bFGF, PDGF and others activate endothelial cells via stimulation of their cell surface receptors (for example VEGFR1/Flt-1 and VEGFR2/Flk-l/KDR; reviewed in Ferrara, 2004, Endocr. Rev., 25, 581-611). These activated cells undergo a process of cellular proliferation, elevated expression of cell adhesion molecules, increased secretion of proteolytic enzymes and increased cellular migration and invasion. A number of distinct molecules are involved to promote proliferation and invasion, including members of the integrin, selectin and immunoglobulin gene super family for adhesion as well as proteolytic enzymes such as matrix metalloproteinases and serine proteinases for degrading the extracellular matrix (Brooks, 1996, Eur. J. Cancer, 32A, 2423). Finally, a complex cascade of biochemical signals derived from cell surface receptors interacting with extracellular matrix components and soluble factors, leading to lumen formation and differentiation into mature blood vessels.

While little is known about the molecular mechanisms of choroidal and/or retinal neovascularization, it has been shown that said specific angiogenic processes are responsible for the majority of severe vision loss in patients with AMD, as well as patients suffering from other retinopathies, such as diabetic retinopathy or retinopathy of prematurity.

Age-related macular degeneration is the leading cause of blindness in developed countries with approximately 15 million people with the disease in the United States. AMD is characterized as a progressive degenerative disease of the macula. There are two forms of AMD: neovascular and non-neovascular. The non-neovascular form of AMD is more common and leads to a slow deterioration of the macula with a gradual loss of vision over a period of years. The neovascular fort of the disease is responsible for the majority of cases of severe vision loss and is due to proliferation of abnormal blood vessels behind the retina leading to hemorrhage and fibrosis which result in visual abnormalities. Current therapeutic efforts and clinical trials are primarily aimed at halting the growth of the neovascular membrane in AMD, e.g. using angiogenesis inhibitors, laser photocoagulation and/or photodynamic therapy (PDT) (see for example WO2004034889). However, only a fraction of eyes meet to the eligibility criteria for such therapeutic interventions and those treated have a high recurrence rate and low therapeutic benefit. Therefore, despite advances in treatment, AMD is still the most common cause of visual impairment in the developed world.

Another leading cause of blindness in adults between the ages of 20 and 74 years is diabetic retinopathy (DR). Seven million people in the United States have diabetes. While management of diabetic retinopathy has improved as a result of landmark clinical trials, risk of complications, such as loss of visual acuity, loss of night vision and loss of peripheral vision, remains significant and treatment sometimes fails. Diabetic retinopathy is characterized by aberrant neovascularization of the retinal vasculature with edema and breakdown in the blood-retinal barrier (BRB) that leads to hemorrhage, macular oedema, tissue damage and retinal scarring. Unfortunately, current treatment options (e.g. laser photocoagulation) are not fully satisfactory and the disease is often progressive.

An increasing body of evidence indicates that inhibition of different molecules involved in the angiogenic cascade offers the potential to treat probable cause of these neovascularization related disorders, including that of tumoral and ocular tissues, by blocking key mediating steps in disease progression (see for example Shibuya, 2003, Nippon Yakurigaku Zasshi, 122, 498-503; Ferrara, 2004, Endocrine Reviews, 25, 581-611; or US 20060030529). Example of these angiogenic inhibitors, including inhibitors of their related receptor, are known in the art and include, e.g., ZD6474 (Tuccillo et al., 2005, Clin Cancer Res., 11, 1268-1276); soluble Tie2 and VEGF-1 receptors (Hangai at al., 2001, Hum Gene Ther., 12, 1311-1321 and Honda et al., 2000, Gene Ther., 7, 978-985, respectively), angiopoietin (especially Ang-2) and PDGF inhibitors; pigment epithelium-derived factor (PEDF) (Rasmussen et al., 2001, Hum Gene Ther., 12, 2029-2032), endostatin (Mori at al., 2001, Am J Pathol., 159, 313-320), and angiostatin (Lai et al., 2001, invest Opthalmol Vis Sci., 42, 2401-2407); tissue inhibitor of metalloprotease-3 (Takahashi et al., 2000, Am J Opthalmol., 130, 774-781); VEGF inhibitory aptamers, e.g., Macugen® (pegaptanib, Pfizer); antibodies or fragments thereof, e.g., anti-VEGF antibodies, e.g., bevacizumab (Avastin®, Genentech), or fragments thereof, e.g., ranihizumab (Lucentis®, Genentech); soluble fins-like tyrosine kinase 1 (sFlt1) polypeptides or polynucleotides (Harris et al., Clin Cancer Res. 2001 July; 7(7):1992-7; U.S. Pat. No. 5,861,484); PTK/ZK which inhibits VEGF signal transduction by blocking the tyrosine kinase (Maier et al., 2005, Graefes Arch. Clin. Exp. Ophthalmol., 243, 593-600); KRN633 (Nakamura at al., 2004, Mol Cancer Ther., 3, 1639-1649); inhibitors of integrins (for example ανβ33 and α5β1); VEGF-Trap® (Regeneron); and Alpha2-antiplasmin (Matsuno et al, Blood 2003; 120:3621-3628). Most of these angiogenic inhibitors are directed towards blocking the initial growth factor mediated activation step induced by vascular endothelial growth factor (VEGF). Therefore, VEGF has been considered as an appealing target for anticancer therapeutics, especially in combination with chemotherapy, radiotherapy or other antiangiogenic agents (see Ferrara, 2005, Oncology, 69, 11-16). Similarly, studies have shown regression or prevention of neovascularization in multiple vascular beds in several animal models, using various types of anti-VEGF agents (e.g. Gragoudas et. al., 2004, N. Engl. J. Med., 351, 2805-2816; Rothen et al., 2005, Opthalmol Olin North Am., 18, 561-567 or Ng et al., 2006, Nat Rev Drug Discov., 5, 123-132) indicating that anti-VEGF therapy is a promising treatment for retinal and/or choroidal neovascularisation related disorders, such as CNV, AMD, diabetic retinopathy (for reviews of VEGF and its inhibitors, see, e.g., Campochiaro, 2004, Expert Opin Biol Ther., 4, 1395-1402; Ferrara, 2004, Endocr. Rev., 25, 581-611; and Verheul and Pinedo, 2008, Drugs Today, 39 Suppl C:81-93).

However, while these results are very encouraging, there is currently no standard and effective therapy for the treatment of neovascularisation and excessive vascular permeability in ocular tissues. Actually, the interest of anti-angiogenic therapy for cancer by inhibition of the vascular endothelial growth factor (VEGF) pathway has been minored by occurence of resistance to anti-VEGF treatment.

Accordingly, existing methods for treating neovascular ocular disease are in need of improvement in their ability to inhibit or eliminate various forms of neovascularization, including retinal and/or choroidal neovascularization, and to treat related disorders. Future efforts must be directed towards the identification of new therapies in order to improve the efficacy of antiangiogenic therapy. The present invention fulfills these needs and further provides other related advantages.

The present invention intends to provide improved compositions and methods for the treatment of ocular neovascularization using compounds that inhibit VEGF in combination with a second therapy. In one aspect of the present invention, there is provided compositions and methods for preventing and treating choroidal and/or retinal neovascularization and related ophthalmic disorders, and more specifically AMD, CNV, retinopathy of prematurity, traumatic eye injury, diabetic retinopathy, some inflammatory ophthalmic disorders (e.g. Birdshot retinochoroidopathy or multifocal choroiditis) and the like.

According to a first embodiment, the Invention provides a combination product comprising a therapeutically effective amount of (i) at least one anti-angiogenesis compound and (ii) at least one corticosteroid, for simultaneous or consecutive administration, or administration which is staggered over time.

This combination product of the Invention is of particular interest for treating and/or preventing ocular pathologies associated with neovascularization.

According to one specific embodiment, said combination product further comprises an ophthalmically compatible solvent component.

As used herein throughout the entire application, the terms “a” and “an” are used in the sense that they mean “at least one”, “at least a first”, “one or more” or “a plurality” of the referenced compounds or steps, unless the context dictates otherwise. More specifically, “at least one” and “one or more” means a number which is one or greater than one, with a special preference for one, two or three.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range.

As used herein, the term “comprising”, “containing” when used to define products, compositions and methods, is intended to mean that the products, compositions and methods include the referenced compounds or steps, but not excluding others.

The term “patient” refers to a vertebrate, particularly a member of the mammalian species and includes, but is not limited to, domestic animals, sport animals, primates including humans. The term “patient” is in no way limited to a special disease status, it encompasses both patients who have already developed a disease of interest and patients who are not sick.

As used herein, the term “treatment” or “treating” encompasses prophylaxis and/or therapy. Accordingly the compositions and methods of the present invention are not limited to therapeutic applications and can be used in prophylaxis ones. Therefore “treating” or “treatment” of a state, disorder or condition includes: (i) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (ii) inhibiting the state, disorder or condition, i.e., arresting or reducing the development of the disease or at least one clinical or subclinical symptom thereof, or (iii) relieving the disease, i.e. causing regression of the state, disorder or condition or at least one of its clinical or subclinical symptoms.

The term “corticosteroid” refers to any naturally occurring or synthetic compound characterized by a hydrogenated cyclopentanoperhydro-phenanthrene ring system and having immunosuppressive and/or antiinflammatory activity.

Naturally occurring corticosteroids are generally produced by the adrenal cortex. Synthetic corticosteroids may be halogenated.

Non limiting examples of corticosteroids are 1′-alpha, 17-alpha,21-trihydroxypregn-4-ene-3,20-dione; 11-beta, 16-alpha, 17,21-tetrahydroxypregn-4-ene-3,20-dione; 11-beta, 16-alpha, 17,21-tetrahydroxypregn-1,4-diene-3,20-dione; 11-beta, 17-alpha,21-trihydroxy-6-alpha-methylpregn-4-ene-3,20-dione; 11-dehydrocorticosterone; 11-deoxycortisol; 11-hydroxy-1,4-androstadiene-3,17-dione; 11-ketotestosterone; 14-hydroxyandrost-4-ene-3,6,17-trione; 15,17-dihydroxyprogesterone; 16-methylhydrocortisone; 17,21-dihydroxy-16-alpha-methylpregna-1,4,9(11)-triene-3,20-dione; 17-alpha-hydroxypregn-4-ene-3,20-dione; 17-alpha-hydroxypregnenolone; 17-hydroxy-16-beta-methyl-5-beta-pregn-9(11)-ene-3,20-dione; 17-hydroxy-4,6,8(14)-pregnatriene-3,20-dione; 17-hydroxypregna-4,9(11)-di-ene-3,20-dione; 18-hydroxycorticosterone; 18-hydroxycortisone; 18-oxocortisol; 21-acetoxypregnenolone; 21-deoxyaldosterone; 21-deoxycortisone; 2-deoxyecdysone; 2-methylcortisone; 3-dehydroecdysone; 4-pregnene-17-alpha, 20-beta, 21-triol-3,11-dione; 6,17,20-trihydroxypregn- -4-ene-3-one; 6-alpha-hydroxycortisol; 6-alpha-fluoroprednisolone, 6-alpha-methylprednisolone, 6-alpha-methylprednisolone 21-acetate, 6-alpha-methylprednisolone 21-hemisuccinate sodium salt, 6-beta-hydroxycortisol, 6-alpha, 9-alpha-difluoroprednisolone 21-acetate 17-butyrate, 6-hydroxycorticosterone; 6-hydroxydexamethasone; 6-hydroxyprednisolone; 9-fluorocortisone; alclomethasone dipropionate; aldosterone; algestone; alphaderm; amadinone; amcinonide; anagestone; androstenedione; anecortave acetate; beclomethasone; beclomethasone dipropionate; betamethasone 17-valerate; betamethasone sodium acetate; betamethasone sodium phosphate; betamethasone valerate; bolasterone; budesonide; calusterone; chlormadinone; chloroprednisone; chloroprednisone acetate; cholesterol; ciclesonide; clobetasol; clobetasol propionate; clobetasone; clocortolone; clocortolone pivalate; clogestone; cloprednol; corticosterone; cortisol; cortisol acetate; cortisol butyrate; cortisol cypionate; cortisol octanoate; cortisol sodium phosphate; cortisol sodium succinate; cortisol valerate; cortisone; cortisone acetate; cortivazol; cortodoxone; daturaolone; deflazacort, 21-deoxycortisol, dehydroepiandrosterone; delmadinone; deoxycorticosterone; deprodone; descinolone; desonide; desoximethasone; dexafen; dexamethasone; dexamethasone 21-acetate; dexamethasone acetate; dexamethasone sodium phosphate; dichlorisone; diflorasone; diflorasone diacetate; diflucortolone; difluprednate; dihydroelatericin a; domoprednate; doxibetasol; ecdysone; ecdysterone; emoxolone; endrysone; enoxolone; fluazacort; flucinolone; flucloronide; fludrocortisone; fludrocortisone acetate; flugestone; flumethasone; flumethasone pivalate; flumoxonide; flunisolide; fluocinolone; fluocinolone acetonide; fluocinonide; fluocortin butyl; 9-fluorocortisone; fluocortolone; fluorohydroxyandrostenedione; fluorometholone; fluorometholone acetate; fluoxymesterone; fluperolone acetate; fluprednidene; fluprednisolone; flurandrenolide; fluticasone; fluticasone propionate; formebolone; formestane; formocortal; gestonorone; glyderinine; halcinonide; halobetasol propionate; halometasone; halopredone; haloprogesterone; hydrocortamate; hydrocortiosone cypionate; hydrocortisone; hydrocortisone 21-butyrate; hydrocortisone aceponate; hydrocortisone acetate; hydrocortisone buteprate; hydrocortisone butyrate; hydrocortisone cypionate; hydrocortisone hemisuccinate; hydrocortisone probutate; hydrocortisone sodium phosphate; hydrocortisone sodium succinate; hydrocortisone valerate; hydroxyprogesterone; inokosterone; isoflupredone; isoflupredone acetate; isoprednidene; loteprednol etabonate; meclorisone; mecortolon; medrogestone; medroxyprogesterone; medrysone; megestrol; megestrol acetate; melengestrol; meprednisone; methandrostenolone; methylprednisolone; methylprednisolone aceponate; methylprednisolone acetate; methylprednisolone hemisuccinate; methylprednisolone sodium succinate; methyltestosterone; metribolone; mometasone; mometasone furoate; mometasone furcate monohydrate; nisone; nomegestrol; norgestomet; norvinisterone; oxymesterone; paramethasone; paramethasone acetate; ponasterone; prednicarbate; prednisolamate; prednisolone; prednisolone 21-diethylaminoacetate; prednisolone 21-hemisuccinate; prednisolone acetate; prednisolone farnesylate; prednisolone hemisuccinate; prednisolone-21 (beta-D-glucuronide); prednisolone metasulphobenzoate; prednisolone sodium phosphate; prednisolone steaglate; prednisolone tebutate; prednisolone tetrahydrophthalate; prednisone; prednival; prednylidene; pregnenolone; procinonide; tralonide; progesterone; oromegestone; rhapontisterone; rimexolone; roxibolone; rubrosterone; stizophyllin; tixocortol; topterone; triamcinolone; triamcinolone acetonide; triamcinolone acetonide 21-palmitate; triamcinolone benetonide; triamcinolone diacetate; triamcinolone hexacetonide; trimegestone; turkesterone; and wortmannin.

The terms “anti-angiogenesis compound”, “angiogenesis inhibitor” are used herein interchangeably to refer to a compound that inhibits angiogenesis (i.e. the growth of new blood vessels).

According to one specific embodiment, said anti-angiogenesis compound is an immunosuppressant compound.

According to one preferred embodiment, said immunosuppressant compound is selected in the group consisting of calcineurin inhibitors and mTOR inhibitors.

According to another specific embodiment, said anti-angiogenesis compound is compound that inhibits VEGF.

As used herein, “compound” refers to any agent, chemical substance, or substrate, whether organic or inorganic, or any protein including antibodies and functional fragments thereof, peptides, polypeptides, peptoids, nucleic acids, oligonucleotides, and the like. Compounds useful in the invention include those described herein in any of their pharmaceutically acceptable forms, including isomers such as diastereomers and enantiomers, salts, esters, solvates, and polymorphs thereof, as well as racemic mixtures and pure isomers of the compounds described herein.

As used herein, “compound that inhibits VEGF” refers to a compound that inhibits the activity or production of vascular endothelial growth factor (VEGF). It refers for example to compounds capable of binding VEGF, including small organic molecules, antibodies or antibody fragments specific to VEGF, peptides, cyclic peptides, nucleic acids, antisense nucleic acids, RNAi, and ribozymes that inhibit VEGF expression at the nucleic acid level. Examples of compounds that inhibits VEGF are nucleic acid ligands of VEGF, such as those described in U.S. Pat. No. 6,168,778 or U.S. Pat. No. 6,147,204, EYE001 (previously referred to as NX1838) which is a modified, pegylated aptamer that binds with high affinity to the major soluble human VEGF isoform; VEGF polypeptides (e.g. U.S. Pat. No. 6,270,933 and WO 99/47677); oligonucleotides that inhibit VEGF expression at the nucleic acid level, for example antisense RNAs (e.g. U.S. Pat. No. 5,710,136; U.S. Pat. No. 5,661,135; U.S. Pat. No. 5,641,756; U.S. Pat. No. 5,639,872; and U.S. Pat. No. 5,639,736). Other examples of inhibitors of VEGF signaling known in the art (see introduction of the present invention) include, e.g., ZD6474 (Tuccillo et al., 2005, Olin Cancer Res., 11, 1268-76); COX-2, Tie2 receptor, angiopoietin, and neuropilin inhibitors; pigment epithelium-derived factor (PEDF), endostatin, and angiostatin, soluble fins-like tyrosine kinase 1 (sFlt1) polypeptides or polynucleotides (Harris et al., 2001, Olin Cancer Res., 7, 1992-1997; U.S. Pat. No. 5,861,484); PTK787/ZK222 584; KRN633 (Maier et al., 2004, Mol Cancer Ther., 3, 1639-1649); VEGF-Trap® (Regeneron); and Alpha2-antiplasmin (Matsuno et al, 2003, Blood, 120, 3621-3628). For reviews of VEGF and its inhibitors, see, e.g., Campochiaro, 2004, Expert Opin Biol Ther., 4, 1395-1402; Ferrara, 2004, Endocr. Rev., 25, 581-611; the content of which are incorporated herein by reference). According to preferred embodiment, compounds that inhibit VEGF are antibodies to, or antibody fragments thereof, or aptamers of VEGF or a related family member such as (VEGF B. I C, D; PDGF). Preferred examples are anti-VEGF antibodies, e.g Avastin™ (also reviewed as bevacizumab, Genentech), or fragments thereof, e.g. Lucentis™ (also reviewed as rhuFAb V2 or AMD-Fab; ranibizumab, Genentech), and other anti-VEGF compounds such as VEGF inhibitory aptamers, e.g., Macugen™ (also reviewed as pegaptanib sodium, anti-VEGF aptamer or EYE001, Pfizer). According to a more preferred embodiment, the compound that inhibits VEGF can further be an immunosuppressant compound, and more preferably is selected in the group consisting of calcineurin inhibitors and mTOR inhibitors.

As used herein, “antibody” encompasses polyclonal and monoclonal antibody preparations, CDR-grafted antibody preparations, as well as preparations including hybrid antibodies, altered antibodies, F(AB)′₂ fragments, F(AB) molecules, Fv fragments, single domain antibodies, chimeric antibodies and functional fragments thereof which exhibit immunological binding properties of the parent antibody molecule. The antibodies can also be humanized. The term “monoclonal antibody” is not limited to antibodies produced through hybridoma technology. The term “monoclonal antibody” refers to an antibody or functional fragment thereof that is derived from a single clone, including any eukaryotic, prokaryotic, or phage clone, and not the method by which it is produced.

Non limiting examples of calcineurin inhibitors are tacrolimus (also named FK-506—Fujisawa Pharma, co), LX211 (also named ISAtx247—Iso Teknika, Inc), ascomycins, pimecrolimus, and cyclosporins and their derivatives, including those listed in compound definition above. According to most preferred embodiment, the calcineurin inhibitor of the present Invention is cyclosporin A. See Wilasrusmee et al., 2005 (Int. Angiol.; 24, 372-379) for example illustrating the antiangiogenesis properties of cyclosporins.

Non limiting examples of mTOR inhibitors are rapamycin (also known as sirolimus, Wyeth), temsirolimus (CCI-779, Wyeth), everolimus, (RAD001, Novartis Pharma AG), and AP23573 (Ariad Pharmaceuticals) and their derivatives, including those listed in compound definition above.

According to another embodiment, said combination product further comprises a biocompatible polymeric or fibrin glue component in an amount effective to delay release of the said compound that inhibits VEGF and/or said corticosteroid, especially into the interior of the eye after the combination product is intraocularly placed in the eye. According to another specific embodiment, said combination product further comprises an ophthalmically compatible solvent component in an amount effective to solubilize the said polymeric or fibrin glue component, the combination product being effective, after being intraocularly placed into the interior of the eye, to form a sustained release of the said compound that inhibits VEGF and/or said corticosteroid in the eye relative to intraocular placement of a substantially identical composition without the polymeric or fibrin glue component.

In another aspect of the invention, the combination product of the invention may further comprise a compound selected in the group consisting of an oestrogen (e.g. oestrodiol), an androgen (e.g. testosterone) retinoic acid derivatives (e.g. 9-cis-retinoic acid, 13-trans-retinoic, acid, all-trans retinoic acid), a vitamin D derivative (e.g. calcipotriol, calcipotriene), a non-steroidal, anti-inflammatory agent, a selective serotonin reuptake inhibitor (SSRI; e.g. fluoxetine, sertraline, paroxetine), a tricyclic antidepressant (TCA; e.g. maprotiline, amoxapine), a phenoxy phenol (e.g. triclosan), an antihistaminine (e.g. loratadine, epinastine), a phosphodiesterase inhibitor (e.g. ibudilast), an anti-infective agent, a protein kinase C inhibitor, a MAP kinase inhibitor, an anti-apoptotic agent, a growth factor, a nutrient vitamin, an unsaturated fatty acid, and/or ocular anti-infective agents, for the treatment of the ophthalmic disorders set forth herein (see for example compounds disclosed in US 2003/0119786; WO 2004/073614; WO 2005/051293; US 2004/0220153; WO 2005/027839; WO 2005/037203; WO 03/0060026). In still other embodiments of the invention, a mixture of these agents may be used. Ocular anti-infective agents that may be used include, but are not limited to penicillins (ampicillin, aziocillin, carbenicillin, dicloxacillin, methicillin, nafcillin, oxacillin, penicillin G, piperacillin, and ticarcillin), cephalosporins (cefamandole, cefazolin, cefotaxime, cefsulodin, ceftazidime, ceftriaxone, cephalothin, and moxalactam), aminoglycosides (amikacin, gentamicin, netilmicin, tobramycin, and neomycin), miscellaneous agents such as aztreonam, bacitracin, ciprofloxacin, clindamycin, chloramphenicol, cotrimoxazole, fusidic acid, imipenem, metronidazole, teicoplanin, and vancomycin), antifungals (amphotericin B, clotrimazole, econazole, fluconazole, flucytosine, itraconazole, ketoconazole, miconazole, natamycin, oxiconazole, and terconazole), antivirals (acyclovir, ethyldeoxyuridine, foscarnet, ganciclovir, idoxuridine, trifluridine, vidarabine, and (S)-1-(3-dydroxy-2-phospho-nyluethoxypropyl) cytosine (HPMPC)), antineoplastic agents (cell cycle (phase) nonspecific agents such as alkylating agents (chlorambucil, cyclophosphamide, mechlorethamine, melphalan, and busulfan), anthracycline antibiotics (doxorubicin, daunomycin, and dactinomycin), cisplatin, and nitrosoureas), antimetabolites such as antipyrimidines (cytarabine, fluorouracil and azacytidine), antifolates (methotrexate), antipurines (mercaptopurine and thioguanine), bleomycin, vinca alkaloids (vincrisine and vinblastine), podophylotoxins (etoposide (VP-16)), and nitrosoureas (carmustine, (BCNU)), and inhibitors of proteolytic enzymes such as plasminogen activator inhibitors. Doses for topical and sub-conjunctival administration of the above agents, as well as intravitreal dose and vitreous half-life may be found in Intravitreal Surgery Principles and Practice, Peyman G A and Shulman, J Eds., 2nd edition, 1994, Appleton-Longe, the relevant sections of which are expressly incorporated by reference herein.

According to another embodiment, said combination product further comprises a pharmaceutically acceptable carrier. Such pharmaceutical carriers can be sterile liquids, such as water and oils, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. Suitable pharmaceutical excipients include starch, glucose, lactose, sucrose, gelatin, malt, rice, sodium stearate, glycerol monostearate, glycerol, propylene, glycol, water, and the like. The combination product, if desired, can also contain minor amounts of wetting or emulsifying agents, or pH buffering agents, or viscosifying agents. Examples of suitable pharmaceutical carriers are described in “Remington's Pharmaceutical Sciences” by E. W. Martin. In a preferred embodiment, the combination product is formulated in accordance with routine procedures as a pharmaceutical composition adapted for injection into the eye. Typically, combination products for injection are solutions in sterile isotonic aqueous buffer. Where necessary, the combination product may also include a solubilizing agent. Generally, the ingredients are supplied either separately or mixed together in unit dosage form, for example, as a dry lyophilized powder or water free concentrate in a hermetically sealed container such as an ampoule or sachet indicating the quantity of active agent. Where the combination product is to be administered by infusion, it can be dispensed with an infusion bottle containing sterile pharmaceutical grade water or saline. Where the combination product is administered by injection, an ampoule of sterile water for injection or saline can be provided so that the ingredients may be mixed prior to administration.

According to the Invention, the compound that inhibits VEGF (e.g. cyclosporin A) in the combination product is present in an amount of less or equal to about 10%, preferably less or equal to about 5%, more preferably less or equal to about 2%, even more preferably less or equal to about 1%. In advantageous embodiment, it is less or equal to about 0.5%, preferably less or equal to about 0.1%, more preferably less or equal to about 0.05%, and even more preferably less or equal to 0.01%. According to special embodiments, the compound that inhibits VEGF is cyclosporin A and its concentration in the combination product is between about 0.001% and about 0.05% (e.g., 0.049%, 0.04%, 0.03%, 0.02%, 0.01%, 0.009%, 0.008%, 0.007%, 0.006%, 0.005%, and 0.001%).

According to the Invention, when the combination product is administered for treating front of the eye diseases the corticosteroid in the combination product is present in an amount of about 0.01% to about 4%, more particularly it is present in an amount of about 0.01% to about 1.0% (e.g., 1.0%, 0.90, 0.8%, 0.7%, 0.6%, 0.50, 0.10, 0.09%, 0.08%, 0.07%, 0.06%, 0.05%, and 0.01%). In special embodiment it is in an amount of about 0.01% to about 0.12%. In advantageous embodiment it is about 0.012%.

According to the Invention, when the combination product is administered for treating back of the eye diseases, the corticosteroid in the combination product is present in an amount of about 0.05 mg to about 2 mg, more specifically of about 0.05 mg to about 1 mg, and even more specifically of about 0.05 to about 0.5 mg.

Recommended dosages for Corticosteroid Dosages are as
follows:

| | Lowest approved | |
|---|---|---|
| | concentration | Lowest standard |
| Ophthalmic | for ophthalmic | recommended |
| corticosteroid | administration | dosage |
| Clocortolone Pivalate | 0.1% | N/A |
| Hydrocortisone | 1.0% | 0.5 μg/3 |
| | | times daily |
| Dexamethasone | 0.1% | 0.05 μg/4-6 |
| | | times daily |
| Fluorometholone | 0.1% | 0.05 μg/2-4 |
| | | times daily |
| Loteprednol Etabonate | 0.2% | 0.1 μg/4 |
| | | times daily |
| Medrysone | 1.0% | 0.5 μg/up to |
| | | every 4 hours |
| Prednisolone Acetate | 0.12% | 0.06 μg/2-4 |
| | | times daily |
| Rimexolone | 1.0% | 0.5 μg/4 |
| | | times daily |

(N/A = Not Available)

Other standard recommended dosages for corticosteroids are provided, e.g., in the Merck Manual of Diagnosis & Therapy (17th Ed. M H Beers et al., Merck & Co.) and Physicians' Desk Reference 2003 (57^{th} Ed. Medical Economics Staff et al., Medical Economics Co., 2002). In one embodiment, the dosage of corticosteroid administered is a dosage equivalent to a prednisolone dosage, as defined herein. For example, a low dosage of a corticosteroid may be considered as the dosage equivalent to a low dosage of prednisolone.

According to the present invention, concentrations of corticosteroids can be either the lowest approved concentration (see table above), or 95% or less of the lowest approved concentration. For example, low concentration of corticosteroids of the invention can be 90%, 85%, 80%, 70%, 60%, 50%, 25%, 10%, 5%, 2%, 1%, 0.5% or 0.1% of the lowest approved concentration.

For ophthalmic administration for example, a low concentration of clocortolone pivalate is between 0.01% and 0.1% (e.g., 0.1%, 0.09%, 0.08%, 0.07%, 0.06%, 0.05%, and 0.01%), a low concentration of hydrocortisone is between 0.01% and 1.0% (e.g., 1.0%, 0.9%, 0.8%, 0.7%, 0.6%, 0.5%, 0.1%, 0.09%, 0.08%, 0.07%, 0.06%, 0.05%, and 0.01%), a low concentration of dexamethasone is between 0.01% and 0.1% (e.g., 0.1%, 0.09%, 0.08%, 0.07%, 0.06%, 0.05%, and 0.01%), a low concentration of fluorometholone is between 0.01% and 0.1% (e.g., 0.1%, 0.09%, 0.08%, 0.07%, 0.06%, 0.05%, and 0.01%), a low concentration of loteprednol etabonate is between 0.01% and 0.2% (e.g., 0.2%, 0.1%, 0.09%, 0.08%, 0.07%, 0.06%, 0.05%, and 0.01%), a low concentration of medrysone is between 0.01% and 1.0% (e.g., 1.0%, 0.9%, 0.8%, 0.7%, 0.6%, 0.0.1%, 0.09%, 0.08%, 0.07%, 0.06%, 0.05%, and 0.01%), a low concentration of rimexolone is between 0.01% and 1.0% (e.g., 1.0%, 0.9%, 0.8%, 0.7%, 0.6%, 0.5%, 0.1%, 0.09%, 0.08%, 0.07%, 0.06%, 0.05%, and 0.01%), and a low concentration of prednisolone is between 0.01% and 0.12% (e.g., 0.12%, 0.1%, 0.09%, 0.08%, 0.07%, 0.06%, 0.05%, and 0.01%).

According to one special embodiment, the combination product of the Invention contains 0.012% of prednisolone acetate and 0.05% of cyclosporin.

According to another embodiment, the present invention relates to a method for inhibiting, treating, or preventing neovascularization of ocular tissues, and related disease or condition, in a patient in need of such treatment that comprises the step of administering a combination product of the present invention in said patient.

According to one embodiment, the administration of (i) at least one anti-angiogenesis compound and (ii) at least one corticosteroid, results in a synergistic effect for inhibiting, treating, or preventing the neovascularization of ocular tissues, and related disorders.

According to one special embodiment, the administration of (i) at least one compound that inhibits VEGF compound and (ii) at least one corticosteroid results in a synergistic effect for inhibiting, treating, or preventing the neovascularization of ocular tissues, and related disorders.

According to another special embodiment, the administration of (i) at least one calcineurin inhibitor or/and mTOR inhibitor and (ii) at least one corticosteroid results in a synergistic effect for inhibiting, treating, or preventing the neovascularization of ocular tissues, and related disorders.

According to one preferred embodiment, the administration of (i) at least one cyclosporin, even more preferably cyclosporin A, and (ii) at least one corticosteroid results in a synergistic effect for inhibiting, treating, or preventing the neovascularization of ocular tissues, and related disorders.

As used herein, “patient” is meant any animal having ocular tissue that may be subject to neovascularization. Preferably, the animal is a mammal, which includes, but is not limited to, humans and other primates. The term also includes domesticated animals, such as cows, hogs, sheep, horses, dogs, and cats. The term “patient” is in no way limited to a special disease status, it encompasses both patients who have already developed a disease of interest and patients who are not sick. According to specific embodiment, the patient treated with the combination product of the invention did not experience cell transplantation, and more specifically does not suffer from graft versus host disease (GVHD).

According to the present invention, the method can be used to inhibit, to prevent and to treat a number of diseases and disorders marked by the development of ophthalmic neovascularization and related disorders. According to the present invention, the ophthalmic neovascularization and related disorders thereof (or disease or condition) are for example macular edema, ischemic retinopathy, intraocular neovascularization, age-related macular degeneration (AMD) and more specifically exudative AMD, corneal neovascularization, retinal neovascularization, choroidal neovascularization, retinopathy of prematurity, traumatic eye injury, diabetic macular edema, diabetic retina ischemia, diabetic retinal oedema, proliferative diabetic retinopathy, birdshot disease, multifocal choroiditis and any neovascularization associated with any pathological condition of the eye.

According to the present invention, the administration of compound (i) and compound (ii) of the combination product can be simultaneous or consecutive administration, or administration which is staggered over time. Simultaneously refers to a coadministration. In this case, these two essential compounds [(i) and (ii)] can be mixed to form a composition prior to being administered, or can be administered at the same time to the patient. It is also possible to administer them consecutively, that is to say one after the other, irrespective of which component of the combination product according to the invention is administered first. Finally, it is possible to use a mode of administration which is staggered over time or is intermittent and which stops and restarts at intervals which may or may not be regular. It is pointed out that the routes and sites of administration of the two components can be different. The time interval between the administrations is not critical and can be defined by the skilled person. It is possible to recommend an interval of from 10 min to 72 h, advantageously of from 30 min to 48 h, preferably of from 1 to 24 h and, very preferably, of from 1 to 6 h; but the interval can be larger, and being over month.

Administration of the combination product for ophthalmic applications is preferably by intraocular injection, although other modes of administration may be effective. Typically, ophthalmic composition will be delivered intraocularly (by chemical delivery system or invasive device) to an individual. However, the invention is not limited to intraocular delivery in that it also includes topically (extraocular application) or systemically (e.g. oral or other parenteral route such as for example subcutaneous administration). Parenteral administration is used in appropriate circumstances apparent to the practitioner. Preferably, the ophthalmic compositions are administered in unit dosage forms suitable for single administration of precise dosage amounts.

As mentioned above, delivery to areas within the eye, in situ can be accomplished by injection, cannula or other invasive device designed to introduce precisely metered amounts of a desired ophthalmic composition to a particular compartment or tissue within the eye (e.g. posterior chamber or retina). An intraocular injection may be into the vitreous (intravitreal), or under the conjunctiva (subconjunctival), or behind the eye (retrobulbar), into the sclera, or under the Capsule of Tenon (sub-Tenon), and may be in a depot form. Other intraocular routes of administration and injection sites and forms are also contemplated and are within the scope of the invention. In preferred embodiment the combination product of the invention will be delivered by sub-retinal injection.

In one embodiment, the ophthalmic composition is intraocularly injected (eg, into the vitreous or sub retinal) to treat or prevent an ophthalmic condition. When administering the ophthalmic composition by intraocular injection, the active agents should be concentrated to minimise the volume for injection. Volumes such as this may require compensatory drainage of the vitreous fluid to prevent increases in intraocular pressure and leakage of the injected fluid through the opening formed by the delivery needle. More preferably, the volume injected is between about 1.0 ml and 0.05 ml. Most preferably, the volume for injection is approximately 0.1 ml.

For injection, a concentration less than about 20 mg/ml may be injected, and any amount may be effective depending upon the factors previously described. Preferably a dose of about 10 mg/ml is administered. Sample concentrations include, but are not limited to, about 5 μg/ml to about 50 μg/ml; about 25 μg/ml to about 100 μg/ml; about, 100 μg/ml to about 200 μg/ml; about 200 μg/ml to about 500 μg/ml; about 500 μg/ml to about 750 μg/ml; about 500 μg/ml up to 1 mg/ml etc. preferred 50 mg/ml. The concentration of compound (i) and (ii) can further be different for one said combination product. In preferred embodiment, a maximum of 100 micrograms of compound (ii) is administered.

Intraocular injection may be achieved by a variety of methods well known in the art. For example, the eye may be washed with a sterilising agent such as Betadine® and the compound of the Invention is injected in an appropriate carrier with a fine gauge needle (eg 27 gauge) at a position in the eye such that the compound will settle to the posterior pole towards the ventral surface. It may be necessary to prepare the eye for injection by application of positive pressure prior to injection. In some cases, preliminary vitrectomy may be necessary. Local anaesthetic or general anaesthetic may be necessary.

The syringe used in practicing the method of this invention is suitably one which can accommodate a 21 to 40 gauge needle and is preferably of a small volume, for example 1.5 ml, or more preferably 0.1 ml. Although it is possible that the needle and syringe may be of the type where the needle is removable from the syringe, it is preferred that the arrangement is of a unitary syringe/needle construction. This would clearly limit the possibility of disengagement of the needle from the syringe. It is also preferred that the arrangement be tamper evident. The combination product of the present invention may therefore be provided in the form of a single unit dose, or separated unit doses each containing part of the combination product, in a pre-prepared syringe ready for administration.

A suitable style of syringe is, for example, sold under the name of Uniject® manufactured by Becton Dickinson and Company. In this style of syringe, the material is expelled through the needle into the eye by pressure applied to the sides of a pliable reservoir supplying the needle, rather than by a plunger. As the name implies, the construction of the reservoir and needle forms a single unit.

Topical application of ophthalmic combination product of the invention for the treatment or prevention of ophthalmic disorders may be as ointment, gel or eye drops. The topical ophthalmic composition may further be an in situ gellable aqueous formulation. Such a formulation comprises a gelling agent in a concentration effective to promote gelling upon contact with the eye or with lacrimal fluid in the exterior of the eye. Suitable gelling agents include, but are not limited to, thermosetting polymers such as tetra-substituted ethylene diamine block copolymers of ethylene oxide and propylene oxide (e.g., poloxamine); polycarbophil; and polysaccharides such as gellan, carrageenan (e.g., kappa-carrageenan and iota-carrageenan), chitosan and alginate gums.

The phrase “in situ gellable” as used herein embraces not only liquids of low viscosity that form gels upon contact with the eye or with lacrimal fluid in the exterior of the eye, but also more viscous liquids such as semi-fluid and thixotropic gels that exhibit substantially increased viscosity or gel stiffness upon administration to the eye.

To prepare a topical ophthalmic composition for the treatment of ophthalmic disorders, a therapeutically effective amount of the combination product of the invention is placed in an opthalmological vehicle as is known in the art. For example, topical ophthalmic formulations containing steroids are disclosed in U.S. Pat. No. 5,041,434, whilst sustained release ophthalmic formulations of an ophthalmic drug and a high molecular weight polymer to form a highly viscous gel have been described in U.S. Pat. No. 4,271,143 and U.S. Pat. No. 4,407,792. Further GB 2007091 describes an ophthalmic composition in the form of a gel comprising an aqueous solution of a carboxyvinyl polymer, a water-soluble basic substance and an ophthalmic drug. Alternatively, U.S. Pat. No. 4,615,697, discloses a controlled release composition and method of use based on a bioadhesive and a treating agent, such as an anti-inflammatory agent.

The amount of the combination product to be administered and the concentration of the compound in the topical ophthalmic combination product used in the method depend upon the diluent, delivery system or device, the clinical condition of the patient, the side effects and the stability of the compound in the formulation. Thus, the physician employs the appropriate preparation containing the appropriate concentration of the compounds (i) and/or (ii) and selects the amount of formulation administered, depending upon clinical experience with the patient in question or with similar patients.

As the combination product contains two or more active agents, the active agents may be administered as a mixture, as an admixture, in the same ophthalmic composition, in separate formulations, in extended release formulations, liposomes, microcapsules, or any of the previously described embodiments.

The combination product may be also administered as a slow release formulation, with a carrier formulation such as microspheres, microcapsules, liposomes, etc., as a topical ointment or solution, an intravenous solution or suspension, or in an intraocular injection, as known to one skilled in the art to treat or prevent ophthalmic disorders. By “slow release”, “time-release”, “sustained release” or “controlled release” is meant that the therapeutically active component is released from the formulation at a controlled rate such that therapeutically beneficial levels (but below toxic levels) of the component are maintained over an extended period of time ranging from e.g., about 12 to about 24 hours, thus, providing, for example, a 12 hour or a 24 hour dosage form. A time-release drug delivery system may be administered intraocularly to result in sustained release of the combination product over a period of time. The combination product may be in the form of a vehicle, such as a micro- or macro-capsule or matrix of biocompatible polymers such as polycaprolactone, polyglycolic acid, polylactic acid, polyanhydrides, polylactide-co-glycolides, polyamino acids, polyethylene oxide, acrylic terminated polyethylene oxide, polyamides, polyethylenes, polyacrylonitriles, polyphosphazenes, poly(ortho esters), sucrose acetate isobutyrate (SATE), and other polymers such as those disclosed in U.S. Pat. Nos. 6,667,371; 6,613,355; 6,596,296; 6,413,536; 5,968,543; 4,079,038; 4,093,709; 4,131,648; 4,138,344; 4,180,646; 4,304,767; 4,946,931, each of which is expressly incorporated by reference herein in its entirety, or lipids that may be formulated as microspheres or liposomes. A microscopic or macroscopic ophthalmic composition may be administered through a needle, or may be implanted by suturing within the eye, eg intravitreal cavity or sub-retinal space. Delayed or extended release properties may be provided through various formulations of the vehicle (coated or uncoated microsphere, coated or uncoated capsule, lipid or polymer components, unilamellar or multilamellar structure, and combinations of the above, etc.). The formulation and loading of microspheres, microcapsules, liposomes, etc and their ocular implantation are standard techniques known by one skilled in the art.

The invention also provides a method for the treatment or prophylaxis of ophthalmic disorders related to neovascularisation, said method comprising the step of administering a combination product of the Invention in a biocompatible, biodegradable matrix, for example in the form of a gel or polymer which is preferably suited for insertion into the retina or into a cavity of the eye, anterior or posterior, as an implant. In the case that the combination product is delivered as an implant, it may be incorporated in any known biocompatible biodegradable matrix as a liquid, or in the form, for example, of a micelle using known chemistry or as microparticles.

Slow or extended-release delivery systems include any of a number of biopolymers (biological-based systems), systems employing liposomes, colloids, resins, and other polymeric delivery systems or compartmentalized reservoirs, can be utilized with the compositions described herein to provide a continuous or long term source of therapeutic compound.

In any slow release device prepared, the said compounds (i) and/or (ii) are preferably present in an amount of about 10% to 90% by weight of the implant. More preferably, the said compounds (i) and/or (ii) are from about 50% to about 80% by weight of the implant. In a preferred embodiment, the said compounds (i) and/or (ii) are about 50% by weight of the implant. In a particularly preferred embodiment, the said compounds (i) and/or (ii) are about 70% by weight of the implant.

In one form, implants used in the method of the present invention are formulated with compounds (i) and/or (ii) entrapped within the bio-erodible polymer matrix. Release of the compounds is achieved by erosion of the polymer followed by exposure of previously entrapped compound to the vitreous, and subsequent dissolution and release of compound. The release kinetics achieved by this form of drug release are different than that achieved through formulations which release drug through polymer swelling, such as with hydrogels such as methylcellulose. In that case, the active compound is not released through polymer erosion, but through polymer swelling, which releases active compound as liquid diffuses through the pathways exposed. The parameters which determine the release kinetics include the size of the active compound particles, the water solubility of the active compound, the ratio of active compound to polymer, the method of manufacture, the surface area exposed, and the erosion rate of the polymer.

Exemplary biocompatible, non-biodegradable polymers of particular interest include polycarbamates or polyureas, particularly polyurethanes, polymers which may be cross-linked to produce non-biodegradable polymers such as cross-linked polyvinyl acetate) and the like. Also of particular interest are ethylene-vinyl ester copolymers having an ester content of 4% to 80% such as ethylene-vinyl acetate (EVA) copolymer, ethylene-vinyl hexanoate copolymer, ethylene-vinyl propionate copolymer, ethylene-vinyl butyrate copolymer, ethylene-vinyl pentantoate copolymer, ethylene-vinyl trimethyl acetate copolymer, ethylene-vinyl diethyl acetate copolymer, ethylene-vinyl 3-methyl butanoate copolymer, ethylene-vinyl 3-3-dimethyl butanoate copolymer, and ethylene-vinyl benzoate copolymer.

Additional exemplary naturally occurring or synthetic non-biodegradable polymeric materials include poly(methylmethacrylate), poly(butylmethacrylate), plasticized poly(vinylchloride), plasticized poly(amides), plasticized nylon, plasticized soft nylon, plasticized poly(ethylene terephthalate), natural rubber, silicone, poly(isoprene), poly(isobutylene), poly(butadiene), poly(ethylene), poly(tetrafluoroethylene), poly(vinylidene chloride), polyacrylonitrile, cross-linked poly(vinylpyrrolidone), poly(trifluorochloroethylene), chlorinated poly(ethylene), poly(4,4′-isopropylidene diphenylene carbonate), vinylidene chloride-acrylonitrile copolymer, vinyl chloridediethyl fumarate copolymer, silicone, silicone rubbers (especially the medical grade), poly(dimethylsiloxanes), ethylene-propylene rubber, silicone-carbonate copolymers, vinylidene chloride-vinyl chloride copolymer, vinyl chloride-acrylonitrile copolymer, vinylidene chloride-acrylonitrile copolymer, poly(olefins), poly(vinyl-olefins), poly(styrene), poly(halo-olefins), poly(vinyls), poly(acrylate), poly(methacrylate), poly(oxides), poly(esters), poly(amides), and poly(carbonates).

Diffusion of the active compounds (i) and/or (ii) from the implant may also be controlled by the structure of the implant. For example, diffusion of the compounds (i) and/or (ii) from the implant may be controlled by means of a membrane affixed to the polymer layer comprising the drug. The membrane layer will be positioned intermediate to the polymer layer comprising the compounds (i) and/or (ii) and the desired site of therapy. The membrane may be composed of any of the biocompatible materials indicated above, the presence of agents in addition to the compounds (i) and/or (ii) present in the polymer, the composition of the polymer comprising the compounds (i) and/or (ii), the desired rate of diffusion and the like.

The skilled reader will appreciate that the duration over which any of the ophthalmic combination product used in the method of the invention will dwell in the ocular environment will depend, inter alia, on such factors as the physicochemical and/or pharmacological properties of the compounds employed in the formulation, the concentration of the compound employed, the bioavailability of the compound, the disease to be treated, the mode of administration and the preferred longevity of the treatment. Where that balance is struck will often depend on the longevity of the effect required in the eye and the ailment being treated.

The frequency of treatment according to the method of the invention is determined according to the disease being treated, the deliverable concentration of the compounds (i) and/or (ii) and the method of delivery. If delivering the combination product by intravitreal injection, the dosage frequency may be monthly. Preferably, the dosage frequency is every three months. The frequency of dosage may also be determined by observation, with the dosage being delivered when the previously delivered combination product is visibly cleared. In general, an effective amount of the compound is that which provides either subjective relief of symptoms or an objectively identifiable improvement as noted by the clinician or other qualified observer.

Ophthalmic combination product prepared for use in the method of the present invention to prevent or treat ophthalmic disorders will preferably have dwell times from hours to many months and possibly years, although the latter time period requires special delivery systems to attain such duration and/or alternatively requires repetitive administrations. Most preferably the combination product for use in the method of the invention will have a dwell time (ie duration in the eye) of hours (i.e. 1 to 24 hours), days (i.e. 1, 2, 3, 4, 5, 6 or 7 days) or weeks (i.e. 1, 2, 3, 4 weeks). Alternatively, the combination product will have a dwell time of at least a few months such as, 1 month, 2 months, 3 months, with dwell times of greater than 4, 5, 6, 7 to 12 months being achievable.

If desired, the method or use of the invention can be carried out alone, or in conjunction with one or more conventional therapeutic modalities (such as photodynamic therapy, laser surgery, laser photocoagulation or one or more biological or pharmaceutical treatments. These methods are well known from the skilled man in the art and widely disclosed in the literature). The use of multiple therapeutic approaches provides the patient with a broader based intervention. In one embodiment, the method of the invention can be preceded or followed by a surgical intervention. In another embodiment, it can be preceded or followed by photodynamic therapy, laser surgery, laser photocoagulation. Those skilled in the art can readily formulate appropriate therapy protocols and parameters which can be used.

The present Invention further concerns a method for improving the treatment of a patient which is undergoing one or more conventional treatment as listed above, which comprises co-treatment of said patient along with a combination product of the present invention.

According to another embodiment, the present invention relates to a method for inhibiting, treating, or preventing an angiogenesis-mediated ophthalmic disease or condition in a patient, comprising administering to said patient an amount effective to inhibit, reduce, or prevent angiogenesis of a combination product comprising (i) at least one anti-angiogenesis compound and (ii) at least one corticosteroid.

According to another embodiment; the present invention relates to a method for inhibiting, treating, or preventing an angiogenesis-mediated ophthalmic disease or condition in a patient, comprising administering to said patient an amount effective to inhibit, reduce, or prevent angiogenesis of a combination product comprising (i) at least one compound that inhibits VEGF compound and (ii) at least one corticosteroid.

According to another embodiment, the present invention relates to a method for inhibiting, treating, or preventing an angiogenesis-mediated ophthalmic disease or condition in a patient, comprising administering to said patient an amount effective to inhibit, reduce, or prevent angiogenesis of a combination product comprising (i) at least one calcineurin inhibitor or/and mTOR inhibitor and (ii) at least one corticosteroid.

According to one preferred embodiment, the present invention relates to a method for inhibiting, treating, or preventing an angiogenesis-mediated ophthalmic disease or condition in a patient, comprising administering to said patient an amount effective to inhibit, reduce, or prevent angiogenesis of a combination product comprising (i) at least one cyclosporin, even more preferably cyclosporin A, and (ii) at least one corticosteroid.

According to another embodiment, the present invention relates to a method for inhibiting, treating, or preventing an angiogenesis-mediated ophthalmic disease or condition in a patient, comprising co-administering to said patient an amount effective to inhibit, reduce, or prevent angiogenesis of (i) at least one anti-angiogenesis compound and (ii) at least one corticosteroid.

According to another embodiment, the present invention relates to a method for inhibiting, treating, or preventing an angiogenesis-mediated ophthalmic disease or condition in a patient, comprising co-administering to said patient an amount effective to inhibit, reduce, or prevent angiogenesis of (i) at least one compound that inhibits VEGF and (ii) at least one agent that results in the enhanced degradation of excess accumulated matrix.

According to another embodiment, the present invention relates to a method for inhibiting, treating, or preventing an angiogenesis-mediated ophthalmic disease or condition in a patient, comprising co-administering to said patient an amount effective to inhibit, reduce, or prevent angiogenesis of (i) at least one calcineurin inhibitor or/and mTOR inhibitor and (ii) at least one corticosteroid.

According to one preferred embodiment, the present invention relates to a method for inhibiting, treating, or preventing an angiogenesis-mediated ophthalmic disease or condition in a patient, comprising co-administering to said patient an amount effective to inhibit, reduce, or prevent angiogenesis of (i) at least one cyclosporin, even more preferably cyclosporin A, and (ii) at least one corticosteroid.

According to another embodiment, the present invention relates to a method to cause regression of neovascularization in a patient, comprising administering to said patient an amount effective of a combination product comprising (i) at least one anti-angiogenesis compound and (ii) at least one agent that results in the enhanced degradation of excess accumulated matrix.

According to another embodiment, the present invention relates to a method to cause regression of neovascularization in a patient, comprising administering to said patient an amount effective of a combination product comprising (i) at least one compound that inhibits VEGF and (ii) at least one agent that results in the enhanced degradation of excess accumulated matrix.

According to another embodiment, the present invention relates to a method to cause regression of neovascularization in a patient, comprising administering to said patient an amount effective of a combination product comprising (i) at least one calcineurin inhibitor or/and mTOR inhibitor and (ii) at least one agent that results in the enhanced degradation of excess accumulated matrix.

According to one preferred embodiment, the present invention relates to a method to cause regression of neovascularization in a patient, comprising administering to said patient an amount effective of a combination product comprising (i) at least one cyclosporin, even more preferably cyclosporin A, and (ii) at least one agent that results in the enhanced degradation of excess accumulated matrix.

According to another embodiment, the present invention relates to a method to cause regression of neovascularization in a patient, comprising co-administering to said patient (i) at least one anti-angiogenesis compound and (ii) at least one agent that results in the enhanced degradation of excess accumulated matrix.

According to another embodiment, the present invention relates to a method to cause regression of neovascularization in a patient, comprising co-administering to said patient (i) at least one compound that inhibits VEGF and (ii) at least one agent that results in the enhanced degradation of excess accumulated matrix.

According to another embodiment, the present invention relates to a method to cause regression of neovascularization in a patient, comprising co-administering to said patient (i) at least one calcineurin inhibitor or/and mTOR inhibitor and (ii) at least one agent that results in the enhanced degradation of excess accumulated matrix.

According to one preferred embodiment, the present invention relates to a method to cause regression of neovascularization in a patient, co-administering to said patient (i) at least one cyclosporin, even more preferably cyclosporin A, and (ii) at least one agent that results in the enhanced degradation of excess accumulated matrix.

As used herein, “to cause regression of neovascularization” means to decrease the amount of neovasculature, especially in the eye, in a subject afflicted with neovascular disease, especially an ocular neovascular disease as defined above.

According to another embodiment, the present invention relates to the use of (i) at least one anti-angiogenesis compound and (ii) at least one corticosteroid for the preparation of a composition useful for the prophylactic or therapeutic treatment of ocular neovascularization and related disorders in a patient, and more specifically those cited above.

According to another embodiment, the present invention relates to the use of (i) at least one compound that inhibits VEGF compound and (ii) at least one corticosteroid for the preparation of a composition useful for the prophylactic or therapeutic treatment of ocular neovascularization and related disorders in a patient, and more specifically those cited above.

According to another embodiment, the present invention relates to the use of (i) at least one calcineurin inhibitor or/and mTOR inhibitor and (ii) at least one corticosteroid for the preparation of a composition useful for the prophylactic or therapeutic treatment of ocular neovascularization and related disorders in a patient, and more specifically those cited above.

According a preferred embodiment, the present invention relates to the use of (i) at least one cyclosporin, even more preferably cyclosporin A, and (ii) at least one corticosteroid for the preparation of a composition useful for the prophylactic or therapeutic treatment ocular neovascularization and related disorders in a patient, and more specifically those cited above.

In other aspects, the invention relates to kits. One kit of the invention includes a container containing (i) at least one anti-angiogenesis compound and a container containing (ii) at least one corticosteroid, and instructions for timing of administration of the compounds. Another kit of the invention includes a container containing (i) at least one compound that inhibits VEGF compound and a container containing (ii) at least one corticosteroid for the preparation and instructions for timing of administration of the compounds. Another kit of the invention includes a container containing (i) at least one calcineurin inhibitor or/and mTOR inhibitor and a container containing (ii) at least one corticosteroid for the preparation and instructions for timing of administration of the compounds. Preferred kit of the invention includes a container containing (i) at least one cyclosporin, even more preferably cyclosporin A, and a container containing (ii) at least one corticosteroid for the preparation and instructions for timing of administration of the compounds. The container may be a single container housing both compound (i) and (ii) together or it may be multiple containers or chambers housing individual dosages of the compounds (i) and (ii), such as a blister pack. The kit also has instructions for timing of administration of the combination product. The instructions would direct the subject to take the combination product or separate compound at the appropriate time. For instance, the appropriate time for delivery of the combination product may be as the symptoms occur. Alternatively, the appropriate time for administration of the combination product may be on a routine schedule such as monthly or yearly. The compounds (i) and (ii) may be administered simultaneously or separately as long as they are administered close enough in time to produce a synergistic response.

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

The present invention is not to be limited in scope by the specific embodiments described herein, which are intended for the purpose of exemplification only. Functionally equivalent products, formulations and methods are clearly within the scope of the invention as described herein.

The invention described herein may include one or more range of values (eg size, concentration etc). A range of values will be understood to include all values within the range, including the values defining the range, and values adjacent to the range which lead to the same or substantially the same outcome as the values immediately adjacent to that value which defines the boundary to the range.

### EXAMPLE

### Evaluation of the Effects of Combination Products of the Invention on VEGF-Induced Vascular Leakage in a Rabbit Model of Blood-Retinal Barrier Breakdown

The aim of this study was to determine the efficacy of combination products of the invention (tested at various concentrations) in reducing vascular leakage in a VEGF-induced blood-retinal barrier breakdown model in the rabbit (Edelman et al., 2005, Experimental Eye Research, 80, 249-258).

The combination tested is a mixture of triamcinolone acetonide (TA) and cyclosporin A (CsA).

Male Fauve de Bourgogne (pigmented) rabbits of approximately 4 months of age and weighing between 2.0 kg and 2.5 kg (CEGAV-FR-61350 Saint Mars-d′Egrenne) were used.

Study Design

Seventy-two (56) pigmented rabbits have been randomly divided into seven (7) groups (8 animals per group).

On Day 0, test combinations and control (50 μl) have been administered by single intravitreal injection into the right eyes (the left eyes have been used as controls and remained untreated).

On Day 5, animals have been treated by a single intravitreal injection of 500 ng rhVEGF165 (50 μl) into the right eyes (test and control groups).

On Day 7,
intravenous injection of sodium fluorescein 47 after the VEGF challenge
measurement of fluorescein leakage in the vitreoretinal compartment of both eyes 1 h after fluorescein injection using non-invasive scanning ocular fluorophotometry
the ratio Rt of vitreoretinal fluorescein contents between the right treated and the left untreated eyes used to evaluate the changes in blood-retinal barrier permeability

Route and Method of Administration

Administrations have been performed on Day 0 in all groups. Animals have been anesthetized by an intramuscular injection of xylazine (7.5 mg/kg) and ketamine (32 mg/kg).

Test combinations and control (50 μl) have been injected into the mid-vitreous of the right eyes using an appropriate needle (26-G needle). After cleaning each eye with betadine, the injections have been made about 3 mm posterior to the limbus in the supratemporal quadrant of the eye. The intravitreal injections have been performed under an operating microscope on dilated eyes (1 drop of Neosynephrine®; phenylephrine 10% and 1 drop of Mydriaticum®; tropicamide 0.5%) instilled 15-20 min before the injection, using a contact lens.

Induction of Vascular Leakage

Increase in retinal vascular permeability has been induced on Day 5 by a single 50 μl intravitreal injection of 500 ng rhVEGF₁₆₅ with carrier protein (diluted in PBS) into the treated eyes of all groups using a 100-μl Hamilton syringe (Edelman et al. ARVO meeting 2003, Fort Lauderdale, Fla.-USA. Invest. Opthalmol. Vis. Sci. 2003; 44: ARVO e-abstract No. 328). This injection has been performed under a microscope on animals anesthetized by an intramuscular injection of a mix of xylazine (7.5 mg/kg) and ketamine (32 mg/kg). The pupil has been dilated before hand (around 15-20 min) with one drop of Neosynephrine® and one drop of Mydriaticum® (see above).

Quantification of Vascular Leakage

On day 7, 47 hours after induction, sodium fluorescein (10% in saline solution 0.9%, 50 mg/kg) has been injected via the marginal ear vein on vigil animals. Measurement of ocular fluorescence has been performed with FM-2 Fluorotron Master ocular photometer on both eyes 1 hour following the fluorescein injection. Rabbits have been anesthetized with intramuscular injection of 32 mg/kg ketamine, 7.5 mg/kg xylazine and pupils dilated with one drop of Neosynephrine® and one drop of Mydriaticum® (see above) 20 minutes prior to the examination. A series of scans of 148 steps (with a step size of 0.25 mm) has been performed from the cornea to the retina along the optical axis.

Study Termination

At the end of the evaluation period (day 7), animals have been euthanized by an intravenous injection of overdosed Dolethal®.

The 7 treated groups (8 rabbits per group) were as follows (doses and percentages are provided):
control, i.e. vehicle alone
TA 400 μG (i.e. TA 0.8%)
TA 135 μG (i.e. TA 0.27%)
TA 75 μG (i.e. TA 0.15%)
CsA 15 μG (i.e. CsA 0.03%)
TA 75 μG+CsA 15 μG (ratio 5) (i.e. TA 0.15%/CsA 0.03%)
TA 135 μG+CsA 1.5 μG (ratio 90) (i.e. TA 0.27%/CsA 0.003%)

The results obtained are summarized in the following table:

| | Inhibition of |
|---|---|
| Treatment | retinal vascular leakage |
| CsA 15 μg | None |
| TA 75 μg | 13% |
| TA 135 μg | 27% |
| TA 75 μg + CsA 15 μg | 50% |
| TA 135 μg + CsA 1.5 μg | 73% |
| TA 400 μg | 96% |

Thus the Inventors have shown that
intravitreal injection of triamcinolone acetonide induced a dose-dependent inhibition of the VEGF-induced retinal vascular leakage; a significant and almost complete protective effect was observed at a 400 μg TA dose;
CsA was devoid of any significant effect;
Triamcinolone acetonide at low subtherapeutic doses (i.e. 75 μg & 135 μg) associated with CsA at specific ratios (i.e. 5 and 90, corresponding to CsA doses of 15 μg and 1.5 μg, respectively) showed a greater effect than TA alone.

Likewise, in one separate study, Triamcinolone acetonide at 75 μg associated with CsA at a ratio of 100 (i.e corresponding to CsA dose of 0.75 μg) showed a greater effect than TA alone (44% versus 10% inhibition of the VEGF-induced retinal vascular leakage respectively.

1.-15. (canceled)

16. A regime or regimen for the treatment of an ocular neovascular disease state in a patient afflicted therewith, comprising administering to such patient, for such period of time as required to elicit the desired pharmaceutical response, whether simultaneously, consecutively or staggered over time, thus effective therapeutic amounts of (i) at least one cyclosporin and (ii) at least one corticosteroid.

17. A regime or regimen for the treatment of an ocular neovascular disease state in a patient afflicted therewith, comprising administering to such patient, for such period of time as required to elicit the desired pharmaceutical response, whether simultaneously, consecutively or staggered over time, thus effective therapeutic amounts of (i) at least one compound that inhibits VEGF in combination with (ii) a second ocular treatment therapy.

18. A regime or regimen for the treatment of an ocular neovascular disease state in a patient afflicted therewith, comprising administering to such patient, for such period of time as required to elicit the desired pharmaceutical response, whether simultaneously, consecutively or staggered over time, thus effective therapeutic amounts of (i) at least one anti-angiogenesis compound and (ii) at least one corticosteroid.

19. A regime or regimen for inhibiting unwanted angiogenesis of ocular tissues, comprising administering to a patient in need of such treatment, for such period of time as required to elicit the desired pharmaceutical response, thus effective therapeutic amounts of (i) at least one compound that inhibits vascular endothelial growth factor (VEGF) and (ii) at least one corticosteroid.

20. A regime or regimen for the treatment of an angiogenesis-mediated ophthalmic disease or condition, comprising administering to a patient in need of such treatment, for such period of time as required to inhibit, reduce or prevent angiogenesis, a thus effective therapeutic amount of a pharmaceutical combination product which comprises (i) at least one calcineurin inhibitor and/or mTOR inhibitor and (ii) at least one corticosteroid.

21. A regime or regimen for the treatment of a choroidal and/or retinal neovascularization ophthalmic disorder, AMD, CNV, retinopathy of prematurity, traumatic eye injury, diabetic retinopathy, an inflammatory ophthalmic disorder, retinochoroidopathy and/or multifocal choroiditis, comprising administering to a patient in need of such treatment, for such period of time as required to elicit the desired pharmaceutical response, thus effective therapeutic amounts of (i) at least one compound that inhibits vascular endothelial growth factor (VEGF) and (ii) at least one corticosteroid.

22. A regime or regimen for the treatment of an ocular neovascularization disease state in a patient afflicted therewith, comprising administering to such patient, for such period of time as required to elicit the desired pharmaceutical response, whether simultaneously, consecutively or staggered over time, thus effective therapeutic amounts of (i) at least one immunosuppressant compound and (ii) at least one corticosteroid.

23. A regime or regimen to effect regression of neovascularization, comprising administering to a patient in need of such treatment, for such period of time as required to elicit the desired pharmaceutical response, whether simultaneously, consecutively or staggered over time, thus effective therapeutic amounts of (i) at least one anti-angiogenesis compound and (ii) at least one agent that enhancedly degrades excess accumulated matrix.

24. A regime or regimen for reducing vascular leakage in a VEGF-induced blood-retinal barrier breakdown in a patient afflicted therewith, comprising administering to such patient, for a period of time as required to elicit the desired pharmaceutical response, whether simultaneously, consecutively or staggered over time, thus effective therapeutic amounts of (i) triamcinolone acetonide and (ii) cyclosporin A.

25. A combination product useful for the treatment of an ocular neovascular disease state, whether for simultaneous or consecutive administration, or administration staggered over time, comprising thus effective therapeutic amounts of (i) at least one anti-angiogenesis compound and (ii) at least one corticosteroid.

26. The combination product as defined by claim 25, wherein said at least one anti-angiogenesis compound comprises an immunosuppressant compound.

27. The combination product as defined by claim 26, wherein said at least one immunosuppressant compound is selected from the group consisting of calcineurin inhibitors and mTOR inhibitors.

28. The combination product as defined by claim 25, wherein said at least one anti-angiogenesis compound inhibits VEGF.

29. The combination product as defined by claim 27, comprising the calcineurin inhibitor cyclosporin A.

30. The combination product as defined by claim 25, further comprising an ophthalmically compatible solvent component.

31. The combination product as defined by claim 25, wherein said at least one anti-angiogenesis compound comprises less or equal to about 10% thereof.

32. The combination product as defined by claim 25, wherein said at least one anti-angiogenesis compound is cyclosporin A and comprises from about 0.001% to about 0.05% thereof.

33. The combination product as defined by claim 25, wherein said at least one corticosteroid comprises about 0.01% to about 4% thereof.

34. The combination product as defined by claim 25, comprising 0.012% of prednisolone acetate and 0.05% of cyclosporin.

35. The combination product as defined by claim 25, formulated for intraocular injection.

36. The combination product as defined by claim 25, formulated for topical or systemic administration.
